(19) United States
(12) Patent Application Publication
(10) Pub. No.: US 2020/0397744 A1
(43) Pub. Date: Dec. 24, 2020

(54) COMPOSITIONS COMPRISING CO-CRYSTALS OF STILBENOIDS AND CANNABINOIDS

(71) Applicant: NEUTRISCI INTERNATIONAL INC., Calgary, Alberta (CA)

(72) Inventors: Glen REHMAN, Calgary (CA); Keith Patrick BUSHFIELD, Calgary (CA)

(73) Assignee: NEUTRISCI INTERNATIONAL INC., Calgary, Alberta (CA)

(21) Appl. No.: 16/968,428

(22) PCT Filed: Feb. 8, 2019
(86) PCT No.: PCT/CA2019/050166

§ 371 Date: Aug. 7, 2020

Related U.S. Application Data
(60) Provisional application No. 62/628,735, filed on Feb. 9, 2018.

(51) Int. Cl. A61K 31/352; A61K 9/48; A61K 9/20; A61K 9/00; A61K 31/05

(52) U.S. Cl. CPC A61K 31/352; A61K 9/4808; A61K 9/2072; A61K 9/2018; A61K 9/2095; A61K 31/05; A61K 9/0053

(57) ABSTRACT

A composition for oral administration comprising a cannabinoid in combination with a stilbenoid or derivative thereof and a solubility enhancing agent is described. Oral dosage forms, in the form of dissolvable tablet or capsule with enhanced bioavailability and processes for preparing them are also disclosed. The processes include lyophilisation of the cannabinoid powder or cannabinoid-stilbenoid co-crystals to form a powder which is pressed into tablets. The powder may be generated from the co-crystals of the cannabinoid with pterostilbene or the powder may be formed by subsequently mixing the lyophilized cannabinoid with the stilbenoid before forming the tablet.

### FIELD OF THE INVENTION

The invention relates to compositions comprising pterostilbene in formulations which improve the dissolution rate and bioavailability of cannabinoid compounds.

### BACKGROUND

Cannabinoids are a group of compounds which are ligands to cannabinoid receptors (CB₁, CB₂) found in the human body. Cannabinoids were originally identified in Cannabis sativa L. Over the last few years, cannabinoids have been reported in the scientific literature to counter the symptoms of a broad range of conditions including multiple sclerosis and other forms of muscular spasm, including uterine and bowel cramps; movement disorders; pain, including migraine headache; glaucoma, asthma, inflammation, insomnia, and high blood pressure. There may also be utility for cannabinoids as an oxytoxic, anxiolytic, anti-convulsive, anti-depressant and anti-psychotic agent, or anti-cancer agent, as well as an appetite stimulant.

Over 60 chemically related compounds, collectively classified as cannabinoids, have been isolated from Cannabis sativa L., and other species and strains of Cannabis including tetrahydrocannabinol (THC), cannabidiol (CBD) and cannabinol (CBN). In addition, various synthetic ligands for cannabinoid receptors have been developed during the last few years. The cannabinoids usually divided into groups of classical cannabinoids, non-classical cannabinoids, aminoalkylindol derivatives and eicosanoids. Classical cannabinoids are isolated from Cannabis sativa L. or they can comprise synthetic analogs of these compounds. Non-classical cannabinoids are bi- or tricyclic analogs of tetrahydrocannabinol (THC) (without the pyran ring); aminoalkylindols form a group which differs structurally substantially from classical and non-classical cannabinoids.

The pharmacological and toxicological studies of cannabinoids have focused mainly on THC (commercially available under the name Dronabinol) which in 1985 was approved by the FDA for the treatment of chemotherapy associated nausea and vomiting, and later for AIDS-associated wasting and anorexia. Dronabinol is a synthetic analog of THC which is marketed in USA as Marinol. In Marinol, THC is dissolved in sesame oil and it is administered orally as a capsule containing 5 or 10 mg of THC.

There continues to be a need for enhancing bioavailability of drugs such as cannabinoids. Efforts to improve bioavailability of cannabinoids using natural compounds recognized as having health benefits are particularly desirable.

### SUMMARY

In accordance with the invention, there is provided a cannabinoid composition in an oral dosage form comprising a cannabinoid in combination with a stilbenoid or a derivative thereof and a solubility enhancing agent.

Another aspect of the invention is a process for preparing an oral dosage form containing a cannabinoid and a stilbenoid or a derivative thereof, the process comprising: mixing and/or bonding a cannabinoid oil with the stilbenoid or the derivative thereof and generating cocrystals of the cannabinoid and the stilbenoid; lyophilizing the cocrystals to form a powder; adding a solubility enhancing agent to the powder; and formulating the powder into the oral dosage form.

Another aspect of the invention is a process for preparing an oral dosage form containing a cannabinoid and a stilbenoid or a derivative thereof, the process comprising: mixing an oil of the cannabinoid with a solubility enhancing agent; lyophilizing the oil to form a powder; adding the stilbenoid or the derivative thereof to the powder; and formulating the powder into the oral dosage form.

In some embodiments of the composition, the composition is in an oral dosage form.

In some embodiments, the cannabinoid is tetrahydrocannabinol (THC), cannabidiol (CBD), or cannabinol (CBN), or any combination thereof.

In some embodiments, the cannabinoid is THC.

In some embodiments, the stilbenoid or derivative thereof is selected from the group consisting of: resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetucleistol E, pinostilbene, pterostilbene, isoharpontigenin, gnetucleistol D, 4-methoxyresveratrol, rhaponticin, and rhapontigenin, cavicularin, 1-hydroxyphenanthrene and juncusol.

In some embodiments, the oral dosage form is configured for sublingual or buccal administration.

In some embodiments, the oral dosage form is a dissolvable tablet or capsule.

In some embodiments, the solubility enhancing agent is a carbohydrate.

In some embodiments, the carbohydrate is mannitol.

In some embodiments, the cannabinoid is present in an amount between about 1 mg to about 20 mg.

In some embodiments, the stilbenoid or derivative thereof is present in an amount between about 10 mg to about 40 mg.

### DETAILED DESCRIPTION

A number of possible alternative features are introduced during the course of this description. It is to be understood that, according to the knowledge and judgment of persons skilled in the art, such alternative features may be substituted in various combinations to arrive at different embodiments of the present invention.

Embodiments of the invention described herein relate to oral dosage forms of co-crystals of stilbenoids or derivatives thereof, and cannabinoids in compositions suitable for sublingual and/or buccal delivery, which have improved dissolution rates and enhanced bioavailability of cannabinoids. Other embodiments relate to forming tablets containing cannabinoids and stilbenoids without forming cocrystals thereof.

The major problem of THC in oral administration is its low bioavailability due to its poor dissolution properties and high first-pass metabolism. The bioavailability of orally ingested THC ranges from only 6% to approximately 20% depending on the drug vehicle employed (see U.S. Pat. No. 8,415,507, incorporated herein by reference in its entirety).

Stilbenoids are hydroxylated derivatives of stilbene and have a C6-C2-C6 structure. They belong to the family of phenylpropanoids and share most of their biosynthesis pathway with chalcones. Stilbenoids have been suggested to have a number of health benefits as antioxidants, antifungals, anti-aging effects, anti-cancer effects, obesity prevention effects, cardiovascular protective effects and protective effects for brain/cognitive mental health. Stilbenoids and derivatives thereof include, but are not limited to resveratrol, piceatannolin, pinosylvin, astringin, piceid, oxyresveratrol, amelopsin A, amelopsin B (resveratrol dimers), vitisin A (resveratrol tetramer) dihydrostilbenoids (combretastatin, combretastatin B-1, isonotholaenic acid) O-methylated stilbenoids (combretastatin A-1, combretastatin A-4, gnetucleistol E, pinostilbene, pterostilbene, isoharpontigenin, oligostilbenoids, phenanthrenoids, gnetucleistol D, 4-methoxyresveratrol, rhaponticin, and rhapontigenin), oligostilbenoids, phenanthrenoids)cavicularin, 1-hydroxyphenanthrene and juncusol).

Pterostilbene (trans-3,5-dimethoxy-4′-hydroxystilbene) is a naturally occurring stilbenoid compound, and a non-ionizable methylated structural analog of resveratrol. It has been characterized as a nutraceutical, being found in nature in a number of tree barks and a variety of berries, including grapes, as well as plants commonly used in traditional folk medicine. Both resveratrol and pterostilbene have been reported to exhibit a range of biological activities including anti-cancer, antioxidant, anti-inflammatory and other potential health benefits. A number of in vitro and in vivo studies of pterostilbene have been conducted in which it was found to demonstrate cytotoxic activity against cancer cell lines in vitro and decreased plasma glucose levels by 42% in hyperglycemic rats (comparable to the commercially available drug, metformin, which reduces glucose levels by 48%). Additionally, the LDL/HDL cholesterol ratio was significantly lowered in hypercholesterolemic hamsters that were fed 25 ppm pterostilbene in their diet compared to the control animals. The use of pterostilbene to ameliorate oxidative stress and improve working memory and compositions containing pterostilbene are described in published U.S. Patent Application US 20090069444, which is incorporated herein by reference in its entirety. Significant interest in pterostilbene has therefore been generated in recent years due to its perceived health benefits, leading to increased consumption of foods that contain the compound, such as grapes and berries.

A number of pharmacological studies have been conducted on pterostilbene. However, very little investigation on the behavior of pterostilbene in the solid state has appeared in the open literature, and thus its solid-state properties appear not to have been thoroughly studied to date.

Pterostilbene has been noted to have poor solubility in water, making it difficult to incorporate in food extracts or supplements. In addition, pterostilbene exhibits poor bioavailability and is easily oxidized by various enzymes. Efforts to improve the solubility of pterostilbene have focused on formulation approaches such as by using cyclodextrins (U.S. Pat. No. 7,592,328, incorporated herein by reference in its entirety). A number of additional US patents have described physicochemical properties and biological activities of pterostilbene (see for example U.S. Pat. Nos. 8,524,782, 8,133,917, 8,252,845, 8,809,400, 8,841,350, 8,227,510, 9,028,887, and 9,439,875, each of which is incorporated herein by reference in its entirety). Additionally, U.S. Pat. No. 9,474,725, incorporated herein by reference in its entirety, describes compositions infused with lipophilic active agents to provide enhanced bioavailability.

Polymorphic forms of pterostilbene have recently been reported. Five polymorphs of pterostilbene are disclosed in WO/2010/141107, incorporated herein by reference in its entirety.

Due to the development of the drug discovery strategy over the last 20 years, physicochemical properties of drug development candidates have changed significantly. The term “drug” as used herein is also meant to include nutraceuticals and active nutraceutical ingredients, even though nutraceuticals are not subject to regulatory trials and approval. The development candidates are generally more lipophilic and less water soluble, which creates huge problems for the industry. Research has shown that some drug candidates fail in the clinical phase due to poor human bioavailability and/or problems with their formulation. Traditional methods to address these problems, without completely redesigning the molecule, include salt selection, producing amorphous material, particle size reduction, prodrugs, and different formulation approaches.

Although therapeutic or clinical efficacy is the primary concern for a drug (or an active nutraceutical ingredient), the salt and solid-state form (i.e., the crystalline or amorphous form) of a drug candidate can be critical to its pharmacological properties and to its development as a viable drug. Crystalline forms of drugs have been used to alter the physicochemical properties of a particular drug. Each crystalline form of a drug candidate can have different solid-state (physical and chemical) properties which may be relevant for drug delivery. Crystalline forms often have better chemical and physical properties than corresponding non-crystalline forms such as the amorphous form. The differences in physical properties exhibited by a novel solid form of a drug (such as a cocrystal or polymorph of the original drug) affect pharmaceutical parameters such as storage stability, compressibility and density (relevant for formulation and product manufacturing), and dissolution rates and solubility (relevant factors in achieving suitable bioavailability).

Dissolution rates of an active ingredient in vivo (e.g., gastric or intestinal fluid) may have therapeutic consequences since it affects the rate at which an orally administered active ingredient may reach the patient's bloodstream. In addition, solubility, a thermodynamic parameter, is a relevant property in evaluating drug delivery because a poorly soluble crystalline form of a drug will deliver less drug than a more soluble one in the same formulation.

Because these practical physical properties are influenced by the solid-state properties of the crystalline form of the drug, they can significantly impact the selection of a compound as a drug, the ultimate pharmaceutical dosage form, the optimization of manufacturing processes, and absorption in the body. Moreover, finding the most adequate solid-state form for further drug development can reduce the time and the cost of that development.

Obtaining suitable crystalline forms of a drug is a necessary stage for many orally available drugs. Suitable crystalline forms possess the desired properties of a particular drug. Such suitable crystalline forms may be obtained by forming a cocrystal between the drug and a coformer. Cocrystals often possess more favorable pharmaceutical and pharmacological properties or may be easier to process than known forms of the drug itself. For example, a cocrystal may have different dissolution and solubility properties than the drug. Further, cocrystals may be used as a convenient vehicle for drug delivery, and new drug formulations comprising cocrystals of a given drug may have superior properties, such as solubility, dissolution, hygroscopicity, and storage stability over existing formulations of the drug.

A cocrystal of a drug (an active nutraceutical ingredient or an active pharmaceutical ingredient) is a distinct chemical composition between the drug and coformer, and generally possesses distinct crystallographic and spectroscopic properties when compared to those of the drug and coformer individually. Unlike salts, which possess a neutral net charge, but which are comprised of charge-balanced components, cocrystals are comprised of neutral species. Thus, unlike a salt, one cannot determine the stoichiometry of a cocrystal based on charge balance. Indeed, one can often obtain cocrystals having stoichiometric ratios of drug to coformer of greater than or less than 1:1. The stoichiometric ratio of an active pharmaceutical ingredient to coformer is a generally unpredictable feature of a cocrystal.

Without limiting the present invention to any particular definitional construct because others may define the term differently, the term “cocrystals” may be thought of as multi-component crystals composed of neutral molecules. These multi-component assemblies are continuing to excite and find usefulness, particularly within the pharmaceutical arena, for their ability to alter physicochemical properties. More specifically, cocrystals have been reported to alter aqueous solubility and/or dissolution rates, increase stability with respect to relative humidity, and improve bioavailability of active pharmaceutical ingredients.

A necessary consideration when designing cocrystals, if the end goal is a potential marketed drug-product, is incorporating a suitable cocrystal former (coformer) with an acceptable toxicity profile. Within the pharmaceutical industry, coformers are typically selected from the same list of pharmaceutically accepted salt formers, generally regarded as safe (GRAS) and/or everything added to food in the United States (EAFUS) lists, due to previous occurrence of these molecules in FDA approved drug or food products. An additional group of molecules to be considered as possible coformers are the naturally occurring compounds, nutraceuticals.

A nutraceutical (portmanteau of nutrition and pharmaceutical) compound is defined as, “a food (or part of a food) that provides medical or health benefits, including the prevention and/or treatment of a disease and possesses a physiological benefit or reduces the risk of chronic disease.” Utilizing naturally occurring compounds as coformers gives extension to the list of potential molecules accessible to the pharmaceutical industry and provides additional physiological benefits to the consumer.

In some circumstances, such as with cocrystals of carboxylic acids, the coformer is generally viewed as the acid moiety whereas the compound whose therapeutic properties are of interest is viewed as the drug, as in the case of the pterostilbene:glutaric acid cocrystal. In other circumstances, more than one component may be viewed as the drug. In the case of the pterostilbene cocrystals reported herein, one may view pterostilbene as acting as a drug and carbamazepine as a coformer or the reverse. Likewise, one may view the pterostilbene in the pterostilbene:caffeine cocrystal as a drug and the caffeine as a coformer or the reverse.

In a cocrystal, the drug and the coformers each possess unique lattice positions within the unit cell of the crystal lattice. Crystallographic and spectroscopic properties of cocrystals can be analyzed as with other crystalline forms such as with X-ray powder diffraction (XRPD), single crystal X-ray crystallography, and solid-state NMR, among other techniques. Cocrystals often also exhibit distinct thermal behavior compared with other forms of the corresponding drug. Thermal behavior may be analyzed by such techniques as capillary melting point, thermogravimetric analysis (TGA), and differential scanning calorimetry (DSC) to name a few. These techniques can be used to identify and characterize the cocrystals.

A number of cocrystals of pterostilbene have been reported, such as cocrystals of pterostilbene with caffeine, carbamazepine, glutaric acid and piperazine (see U.S. Pat. No. 8,415,507, incorporated herein by reference in its entirety).

Other embodiments of the invention include compositions containing one or more solid forms of the stilbenoid cocrystals such as pharmaceutical or nutraceutical oral dosage forms, including orally dissolvable dosage forms suitable for buccal or sublingual administration. Such pharmaceutical oral dosage forms may include one or more excipients, including, without limitation, binders, fillers, lubricants, emulsifiers, suspending agents, sweeteners, flavorings, preservatives, buffers, wetting agents, disintegrants, effervescent agents, and other conventional excipients and additives. The compositions of the invention can thus include any one or a combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art, which are described in Remington: The Science and Practice of Pharmacy, 21st ed., 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, each of which is incorporated herein by reference in its entirety. These additional formulation additives and agents will often be biologically inactive and can be administered to humans without causing deleterious side effects or interactions.

Suitable additives may include, but are not limited to, microcrystalline cellulose, lactose, sucrose, fructose, glucose, dextrose, sucralose, other sugars, di-basic calcium phosphate, calcium sulfate, cellulose, methylcellulose, cellulose derivatives, kaolin, mannitol, lactitol, maltitol, xylitol, sorbitol, other sugar alcohols, dry starch, dextrin, maltodextrin, other polysaccharides, excipients such as crospovidone, as well as flavoring agents and/or mixtures thereof.

In one embodiment of the present invention the solid-state pterostilbene cocrystal dosage is an oral dosage form. Exemplary oral dosage forms for use in the present disclosure include tablets, capsules, powders, suspensions, and lozenges, which may be prepared by any conventional method of preparing pharmaceutical oral dosage forms. Oral dosage forms, such as tablets, may contain one or more of the conventional, pharmaceutically acceptable additional formulation ingredients, including but not limited to, release modifying agents, glidants, compression aides, disintegrants, effervescent agents, lubricants, binders, diluents, flavors, flavor enhancers, sweeteners, and preservatives. Tablet dosage forms may be partially or fully coated, sub-coated, uncoated, and may include channeling agents. The ingredients are selected from a wide variety of excipients known in the pharmaceutical formulation art. Depending on the desired properties of the oral dosage form, any number of ingredients may be selected alone or in combination for their known use in preparing such dosage forms as tablets.

Pterostilbene, an antioxidant, is known to be beneficial for human health. The invention also provides therapeutic uses of the pterostilbene cocrystals and methods for delivering them, and dosage forms containing them, to humans. The dosage forms may be administered using any amount, any form of pharmaceutical composition and any route of administration effective for the treatment. After formulation with an appropriate pharmaceutically acceptable carrier in a desired dosage, as known by those of skill in the art, the pharmaceutical compositions of this disclosure can be administered to humans and other animals orally, rectally, parenterally, intravenously, intracisternally, intravaginally, intraperitoneally, topically (as by powders, ointments, or drops), bucally, as an oral or nasal spray, or the like, depending on the location and severity of the condition being treated. In one embodiment of the disclosure, the method of delivery is with an oral dosage form.

In certain embodiments, solid forms containing a pterostilbene cocrystal of the invention may be administered at pterostilbene dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired effect. It will also be appreciated that dosages smaller than 0.001 mg/kg or greater than 50 mg/kg (for example 50-100 mg/kg) can be administered to a subject in need thereof.

The pterostilbene cocrystals of the present invention may be characterized by X-ray powder diffraction, thermal gravimetric analysis, differential scanning calorimetry, single crystal X-ray diffraction, and solid state ¹³C NMR. The stability (with respect to relative humidity) and solubility of the pterostilbene cocrystals may be determined as described below.

As used herein, the word “characterize” means to identify a collection of data which may be used to identify a cocrystal of the invention. The process by which cocrystals are characterized involves analyzing data collected on the cocrystals so as to allow one of ordinary skill in the art to distinguish cocrystals of the same active pharmaceutical or nutraceutical ingredient. Chemical identity of cocrystals can often be determined with solution-state techniques such as ¹H NMR spectroscopy which will provide or assist in providing the chemical identity of the coformers as well as the active pharmaceutical ingredient or active nutraceutical ingredient. Thus, such techniques can be used to differentiate and characterize cocrystals having different coformers but the same drug (or active nutraceutical ingredient).

One may also, for example, collect X-ray powder diffraction data on cocrystals such as cocrystals of pterostilbene.

As with any data measurement, there is variability in X-ray powder diffraction data. In addition to the variability in peak intensity, there is also variability in the position of peaks on the x-axis. This variability can, however, typically be accounted for when reporting the positions of peaks for purposes of characterization. Such variability in the position of peaks along the x-axis derives from several sources. One comes from sample preparation. Samples of the same crystalline material, prepared under different conditions, may yield slightly different diffractograms. Factors such as particle size, moisture content, solvent content, and orientation may all affect how a sample diffracts X-rays. Another source of variability comes from instrument parameters. Different X-ray instruments operate using different parameters and these may lead to slightly different diffraction patterns from the same crystalline solid form. Likewise, different software packages process X-ray data differently and this also leads to variability. These and other sources of variability are known to those of ordinary skill in the pharmaceutical arts.

### EXAMPLES

### Example 1: Manufacture of a Cannabinoid-Pterostilbene Composition in Sublingual Tablet Form Using Cocrystals

This example describes a process for manufacture of a composition of a cannabinoid in combination with pterostilbene to enhance the bioavailability of the cannabinoid. In this example embodiment, the cannabinoid is THC.

THC is purified from its source and isolated in the form of an oil according to a conventional process. A reactor vessel is charged with solid pterostilbene and the THC oil in a suitable crystallization solvent. The solution is heated and then cooled to promote formation of cocrystals. The cocrystals are then collected, washed and dried under vacuum.

A solubility-enhancing agent, which may be a carbohydrate such as mannitol for example, is then added to the cocrystals. The cocrystals are then lyophilized to produce a powder suitable for pressing into tablets of sizes appropriate for sublingual administration, according to known tablet-making processes.

### Example 2: Manufacture of a Cannabinoid-Pterostilbene Composition in Sublingual Tablet Form Via Lyophilization of THC Oil and Subsequent Mixing with Pterostilbene

In this example, a sublingual tablet is formed by first mixing THC oil with a solubility enhancing agent such as mannitol first, then lyophilizing the oil to produce a powder. Pterostilbene is then mixed into the powder and the mixture is pressed into tablets of sizes appropriate for sublingual administration, according to known tablet-making processes.

### Example 3: Preliminary Observational Trial of Effectiveness Sublingual Melt Tablets Containing Cannabinoids and Pterostilbene in Pain Management

Two candidate sublingual formulations were prepared containing either THC or CBD in combination with pterostilbene. The THC or CBD oil was heated for about five minutes at about 32° C. Base material containing mannitol, silicone dioxide, sorbitol, crospovidone, microcrystalline cellulose, copovidone, sucralose, and flavoring was mixed with pterostilbene for five minutes at low speed. The heated oil was added to the base material and pterostilbene mixture and mixed at low speed for 5 minutes. The resulting liquid mixture was then placed in stainless steel trays and freeze-dried. The dried mixture was fed into a granulator to generate a granulated mixture which was then pressed into sublingual melt tablets, each containing 5 mg of cannabinoid (THC or CBD), 25 mg of pterostilbene, 800 mg of mannitol, 10 mg of sorbitol, 2 mg of crospovidone, 3 mg of microcrystalline cellulose, 2 mg of sucralose and 100 mg of a flavoring agent.

For the purposes of a preliminary observational trial of the effectiveness of the sublingual melt tablets in pain management four individuals with chronic pain of varying origin were invited to self-administer THC or CBD tablets prepared with the formulation described above. The results were recorded by each of the individuals in a questionnaire and are presented in Tables 1A and 1B below. Individuals 1 and 2 self-administered sublingual tablets containing the CBD formulation and Individuals 3 and 4 self-administered sublingual tablets containing the THC formulation.

TABLE 1A
Observational Clinical Trial Results

| | Medical | | | Pain |
|---|---|---|---|---|
| | Condition/Pain | Dosage | | Improvement |
| Individual | Origin | Period | Tablets/day | Noted |
| 1 | arthritis, | 5 days | 2 | yes |
| | cartilage loss | | | |
| 2 | knee, hip, | 1 day | 2 | yes |
| | neck pain | | | |
| 3 | anxiety, | 1 day | 1 | yes |
| | migraines | | | |
| 4 | muscular | 1 day | 2 | yes |
| | pain | | | |

TABLE 1B
Observational Clinical Trial Results

| | Overall | Experience | Likely to | Likely to |
|---|---|---|---|---|
| Individual | Taste | Rating | Purchase | Recommend |
| 1 | satisfied | satisfied | likely | likely |
| 2 | satisfied | satisfied | definitely | definitely |
| 3 | satisfied | satisfied | definitely | very likely |
| 4 | satisfied | satisfied | definitely | definitely |

The results of this preliminary trial indicate that the tested formulations were generally effective at providing pain management with a satisfactory ratings in overall taste and experience. It can therefore be reasonably predicted that self-administration of a sublingual tablet containing a cannabinoid in combination with a stilbenoid can provide a highly convenient and non-intrusive means of pain management in comparison with self-administration of cannabinoids via other routes.

### Equivalents and Scope

Other than described herein, or unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Unless otherwise defined, all technical and scientific terms used herein have the same meaning as commonly understood by one of ordinary skill in the art to which this invention belongs.

While this invention has been particularly shown and described with references to embodiments thereof, it will be understood by those skilled in the art that various changes in form and details may be made therein without departing from the scope of the invention encompassed by the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

It is also noted that the term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where the term “about” is used, it is understood to reflect+/−10% of the recited value. In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

1. A composition comprising a cannabinoid in combination with a stilbenoid or derivative thereof and a solubility enhancing agent.

2. The composition of claim 1, which is in an oral dosage form.

3. The composition of claim 1, wherein the cannabinoid is tetrahydrocannabinol (THC), cannabidiol (CBD), or cannabinol (CBN), or any combination thereof.

4. The composition of claim 1, wherein the cannabinoid is THC.

5. The composition of claim 1, wherein the stilbenoid or derivative thereof is selected from the group consisting of: resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetucleistol E, pinostilbene, pterostilbene, isoharpontigenin, gnetucleistol D, 4-methoxyresveratrol, rhaponticin, and rhapontigenin, cavicularin, 1-hydroxyphenanthrene and juncusol.

6. The composition of claim 2, wherein the oral dosage form is configured for sublingual or buccal administration.

7. The composition of claim 6, wherein the oral dosage form is a dissolvable tablet or capsule.

8. The composition of claim 1, wherein the solubility enhancing agent is a carbohydrate.

9. The composition of claim 8, wherein the carbohydrate is mannitol.

10. The composition of claim 1, wherein the cannabinoid is present in an amount between about 1 mg to about 20 mg.

11. The composition of claim 1, wherein the stilbenoid or derivative thereof is present in an amount between about 10 mg to about 40 mg.

12. A process for preparing an oral dosage form containing a cannabinoid and a stilbenoid or a derivative thereof, the process comprising:
a) mixing and/or bonding a cannabinoid oil with the stilbenoid or the derivative thereof and generating cocrystals of the cannabinoid and the stilbenoid;
b) lyophilizing the cocrystals to form a powder;
c) adding a solubility enhancing agent to the powder; and
d) formulating the powder into the oral dosage form.

13. The process of claim 12, wherein the cannabinoid is tetrahydrocannabinol (THC), cannabidiol (CBD), or cannabinol (CBN), or any combination thereof.

14. The process of claim 12, wherein the cannabinoid is THC.

15. The process of claim 12, wherein the stilbenoid or derivative thereof is selected from the group consisting of: resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetucleistol E, pinostilbene, pterostilbene, isoharpontigenin, gnetucleistol D, 4-methoxyresveratrol, rhaponticin, and rhapontigenin, cavicularin, 1-hydroxyphenanthrene and juncusol.

16. The process of claim 12, wherein the oral dosage form is configured for sublingual or buccal administration.

17. The process of claim 16, wherein the oral dosage form is a dissolvable tablet or capsule.

18. The process of claim 12, wherein the solubility enhancing agent is a carbohydrate.

19. The process of claim 18, wherein the carbohydrate is mannitol.

20. A process for preparing an oral dosage form containing a cannabinoid and a stilbenoid or a derivative thereof, the process comprising, the process comprising:
a) mixing an oil of the cannabinoid with a solubility enhancing agent;
b) lyophilizing the oil to form a powder;
c) adding the stilbenoid or the derivative thereof to the powder; and
d) formulating the powder into the oral dosage form.

21. The process of claim 20, wherein the cannabinoid is tetrahydrocannabinol (THC), cannabidiol (CBD), or cannabinol (CBN), or any combination thereof.

22. The process of claim 20, wherein the cannabinoid is THC.

23. The process of claim 20, wherein the stilbenoid or derivative thereof is selected from the group consisting of: resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetucleistol E, pinostilbene, pterostilbene, isoharpontigenin, gnetucleistol D, 4-methoxyresveratrol, rhaponticin, and rhapontigenin, cavicularin, 1-hydroxyphenanthrene and juncusol.

24. The process of claim 20, wherein the oral dosage form is configured for sublingual or buccal administration.

25. The process of claim 24, wherein the oral dosage form is a dissolvable tablet or capsule.

26. The process of claim 20, wherein the solubility enhancing agent is a carbohydrate.

27. The process of claim 26, wherein the carbohydrate is mannitol.
